(19) United States
(12) Patent Application Publication
(10) Pub. No.: US 2017/0202896 A1
(43) Pub. Date: Jul. 20, 2017

(54) Cannabis Separation

(71) Applicant: Kevin Anthony Hugh, Osoyoos (CA)

(72) Inventor: Kevin Anthony Hugh, Osoyoos (CA)

(21) Appl. No.: 15/017,674

(22) Filed: Feb. 7, 2016

Related U.S. Application Data
(63) Continuation of application No. 15/000,016, filed on Jan. 18, 2016.

(30) Foreign Application Priority Data
Jan. 25, 2016 (CA) 2919034

(51) Int. Cl. A61K 36/185; B02C 23/08; B03B 1/00; A61K 31/352; A61K 31/05

(52) U.S. Cl. CPC A61K 36/185; A61K 31/352; A61K 31/05; B03B 1/00; B02C 23/08; A61K 2236/17; A61K 2236/15; A61K 2236/30

(57) ABSTRACT

Provided are a cannabis composition in pellet form, a method of preparing a cannabis composition in pellet form, a method of separating a plant resin from a harvested plant material, a method of treatment with a cannabis composition in pellet form, and a kit including a cannabis composition in pellet form. For example, the cannabis composition in pellet form may include a cannabinoid resin. The cannabinoid resin may include one or more of: a tetrahydrocannabinol extract and a cannabidiol extract.

### CROSS-REFERENCE TO RELATED APPLICATIONS

This application claims priority to U.S. patent application Ser. No. 15/000,016, filed on Jan. 18, 2016, and Canadian Pat. App. No. 2,919,034, filed Jan. 25, 2016, each of which are entirely incorporated by reference herein.

### BACKGROUND

The medicinal properties of marijuana have been reported as early as 2700 BC, though the use of marijuana has since remained a controversial topic. Despite marijuana's divided history, twenty-three U.S. states have legalized medicinal marijuana since 1996, and it is likely that more will follow suit, not to mention countries outside the US. Although it is becoming common for patients to request medicinal marijuana to aid in treatment of various ailments, many medical practitioners remain hesitant to prescribe medicinal marijuana due to concerns with reliable and consistent dosing, e.g., due to the uncertainty of the source and particulars of the Cannabis crop. Unfortunately, patients who do not receive prescriptions often turn to the dangerous and illegal recreational market to fulfill their needs. The Cannabis flower, which is typically smoked by users, contains a variety of potentially harmful chemicals and may vary greatly in tetrahydrocannabinol (THC) content. Additionally, the high temperatures (500° C.-700° C.) used to burn and smoke Cannabis plant material are unsafe. Medical practitioners simply feel uncomfortable prescribing a largely unregulated substance and believe it is their responsibility to ensure the safety of their patients.

Furthermore, Cannabis plants have been bred upward over the last few decades to produce crops that contain more than three times the amount of THC than previously grown crops. These designer crops often have lesser amounts of other cannabinoid components, such as cannabidiol (CBD). As with THC, CBD has shown promise as a therapeutic agent and studies have shown that CBD may be safer. The evolution of the Cannabis crop has created the need for highly specific growing environments which inhibits centralized mass production, leading to larger numbers of producers, which may increase cost, e.g., via attempts to maintain compliance with regulation.

The present application appreciates that providing medicinal marijuana in a regulated, standardized manner may be a challenging endeavor.

### SUMMARY

In one embodiment, a method for separating a plant resin from a harvested plant material is provided. The method may include providing the harvested plant material. The harvested plant material may include the plant resin. The method may include at least partly dehydrating the harvested plant material. The method may include cooling the harvested plant material to a process temperature at or below a resin solidification temperature. The process temperature may be effective to provide the plant resin in a solidified state. At the process temperature, the method may include forming the harvested plant material into a plant material aggregate. The plant material aggregate may include the solidified plant resin and plant matter. At the process temperature, the method may include subjecting the plant material aggregate to mechanical separation conditions effective to separate at least a portion of the solidified plant resin from the plant matter.

In another embodiment, an apparatus for separating a plant resin from a harvested plant material is provided. The apparatus may include a separation chamber. The apparatus may include a cooling mechanism operatively coupled to cool the separation chamber and contents thereof to a process temperature. The apparatus may include a mechanical separation mechanism. The separation chamber and the mechanical separation mechanism may be operatively coupled to separate a plant aggregate into a solidified plant resin and plant matter.

In one embodiment, a cannabis composition is provided. The cannabis composition may include a cannabinoid resin. The cannabinoid resin may include one or more of: between about 50% and about 100% (w/w) of a tetrahydrocannabinol extract and between about 0.1% and about 100% (w/w) of a cannabidiol extract. The cannabis composition may be in the form of a compressed pellet.

In another embodiment, a method of preparing a cannabis composition in pellet form is provided. The method may include providing a Cannabis crop. The method may include extracting a cannabinoid resin from the Cannabis crop. The cannabinoid resin may include one or more of: a tetrahydrocannabinol extract and a cannabidiol extract. The method may include formulating the cannabinoid resin into one or more compressed pellets.

In another embodiment, a method of treatment is provided. The method may include providing a patient in need of treatment for a condition. The method may include providing a cannabis composition in the form of a compressed pellet. The method may include administering the compressed pellet to the patient. The administering may be effective to ameliorate one or more symptoms of the condition, whereby the condition is treated.

In another embodiment, a kit is provided. The kit may include a cannabis composition in the form of a compressed pellet. The kit may include instructions directing a user to: provide a patient in need of treatment for a condition, and administer the compressed pellet to the patient.

### BRIEF DESCRIPTION OF THE DRAWINGS

The accompanying figures, which are incorporated in and constitute a part of the specification, illustrate example methods and apparatuses, and are used merely to illustrate example embodiments.

FIG. 1 is a flow diagram describing an example method 100 of preparing a cannabis composition in pellet form;

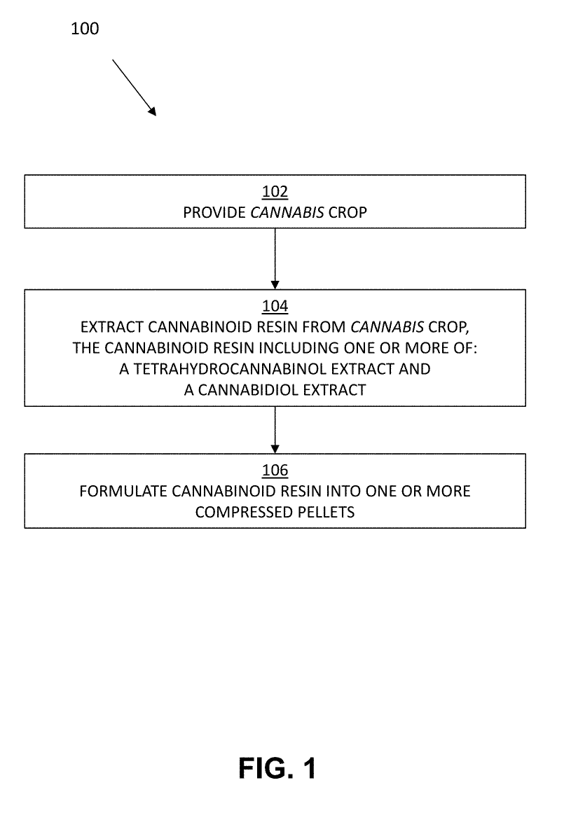

FIG. 2 is a flow diagram describing an example method 200 of separating a plant resin from a harvested plant material;

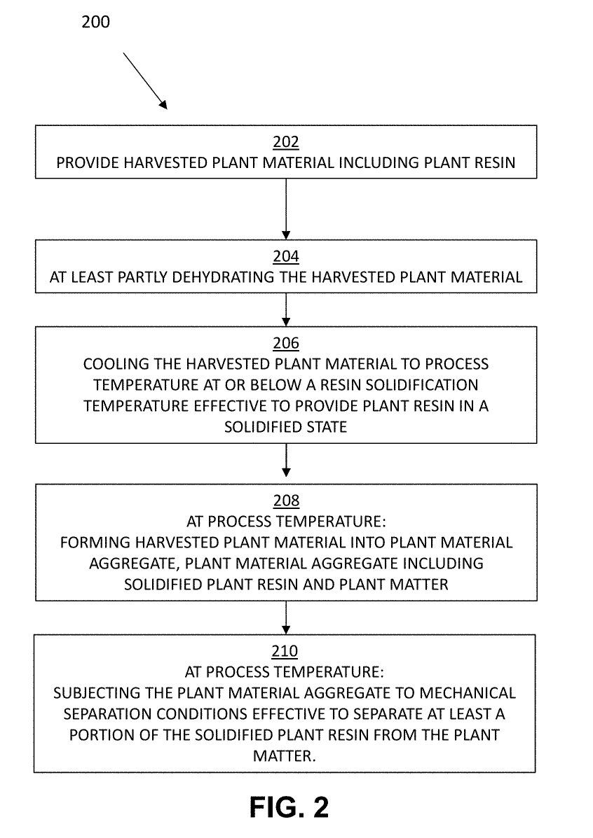

FIG. 3A is a block diagram of an example apparatus for separating a plant resin from a harvested plant material;

FIG. 3B is a schematic showing the operation of an example apparatus for separating a plant resin from a harvested plant material;

FIG. 4 is a flow diagram describing an example method 400 of treatment with a cannabis composition in pellet form; and

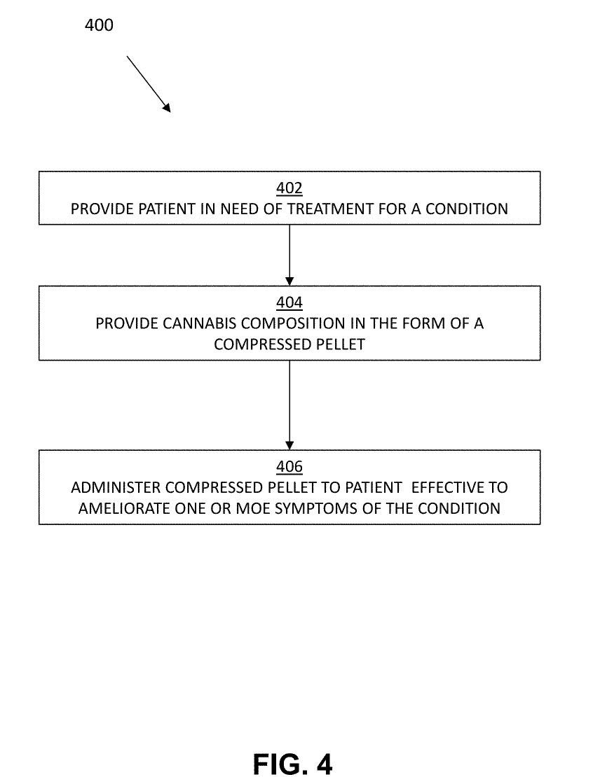

FIG. 5 is a block diagram depicted a kit 500 including a cannabis composition in the form of a compressed pellet.

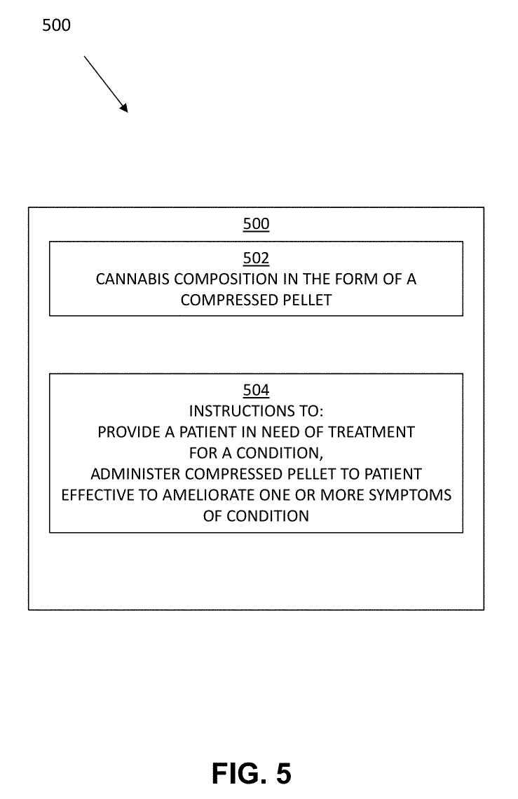

### DETAILED DESCRIPTION

The present application relates to a cannabis composition in a compressed pellet form, a method of preparing a cannabis composition in a compressed pellet form, a method of separating a plant resin from a harvested plant material, a method of treatment with a cannabis composition in a compressed pellet form, and a kit including a cannabis composition in a compressed pellet form. The cannabis composition may permit health practitioners to prescribe reliable and consistent doses of medicinal marijuana products from a renewable source. The cannabis composition may mitigate one or more hazards associated with the use of medicinal marijuana, particularly the concerns of health practitioners regarding consistency of dosing.

In various embodiments, a cannabis composition is provided. The cannabis composition may include a cannabinoid resin. The cannabinoid resin may include one or more of: between about 50% and about 100% (w/w) of a tetrahydrocannabinol extract and between about 0.1% and about 100% (w/w) of a cannabidiol extract. The cannabis composition may be in the form of a compressed pellet.

In some embodiments, the compressed pellet may consist essentially of the cannabinoid resin. In several embodiments, the compressed pellet may consist of the cannabinoid resin.

In several embodiments, the tetrahydrocannabinol extract may be present in the cannabinoid resin in an amount in % (w/w) with respect to the cannabinoid resin of about one or more of: 50, 55, 60, 65, 70, 75, 80, 85, 90, 95, 99, and 100, or a range between any of the preceding values, for example, between about 50 and about 100, between about 60 and about 75, and the like. The tetrahydrocannabinol extract may be present in the cannabinoid resin in less than about 50% (w/w) with respect to the cannabinoid resin. The tetrahydrocannabinol extract may be present in the cannabinoid resin in amount in % (w/w) with respect to the cannabinoid resin of about one or more of: 0, 5, 10, 15, 20, 25, 30, 35, 40, 45 and 50, or a range between any of the preceding values, for example, between about 0 and about 20, between about 35 and about 45, and the like. The cannabinoid resin may be absent of the tetrahydrocannabinol extract.

In some embodiments, the cannabinoid resin may include an amount of cannabidiol extract. The cannabidiol extract may be present in the cannabinoid resin in an amount in % (w/w) with respect to the cannabinoid resin of about one or more of: 0, 5, 10, 15, 20, 25, 30, 35, 40, 45 50, 55, 60, 65, 70, 75, 80, 85, 90, 95, 99, and 100, or a range between any of the preceding values, for example, between about 5 and about 50, between about 25 and about 75, and the like. The cannabinoid resin may be absent of cannabidiol extract.

In several embodiments, the cannabinoid resin may include a tetrahydrocannabinol extract or a cannabidiol extract. For example, the cannabinoid resin may include a tetrahydrocannabinol extract and not include a cannabidiol extract. For example, the cannabinoid resin may include a cannabidiol extract and not include a tetrahydrocannabinol extract. The amount of tetrahydrocannabinol extract may be any amount in % (w/w) with respect to cannabinoid resin as disclosed herein. The amount of cannabidiol extract may be any amount in % (w/w) with respect to cannabinoid resin as disclosed herein. For example, the cannabinoid resin may include an amount of the tetrahydrocannabinol extract between about 50% (w/w) and about 100% (w/w) with respect to the cannabinoid resin. Alternatively, for example, the cannabinoid resin may include an amount of the cannabidiol extract between about 1% (w/w) and about 100% (w/w) with respect to the cannabinoid resin.

In some embodiments, the cannabinoid resin may include a tetrahydrocannabinol extract and a cannabidiol extract. For example, the cannabinoid resin may include an amount of the tetrahydrocannabinol extract between about 50% (w/w) and about 99.9% (w/w) with respect to the cannabinoid resin, and include an amount of the cannabidiol extract between about 0.1% (w/w) and about 50% (w/w) with respect to the cannabinoid resin. The amount of the tetrahydrocannabinol extract and the amount of cannabidiol extract may be any amount in % (w/w) with respect to cannabinoid resin as disclosed herein, as long as the combined amounts of the tetrahydrocannabinol and the cannabidiol extracts do not exceed 100%. For example, the cannabinoid resin may include about 50% tetrahydrocannabinol extract and about 50% cannabidiol extract. For example, the cannabinoid resin may include about 60% tetrahydrocannabinol extract and about 20% cannabidiol extract.

In several embodiments, the compressed pellet may include the cannabinoid resin in an amount in milligrams (mg) of one or more of about: 25, 50, 100, 125, 150, 175, 200, 250, 300, 350, 400, 450, 500, 600, 700, 800, 900, 1000, 1100, 1200, 1300, 1400, 1500, 1600, 1700, 1800, 1900, and 2000, or a range between any of the preceding values, for example, between about 25 mg and about 2000 mg, between about 50 mg and about 500 mg, between about 500 mg and about 1000 mg, between about 200 mg and about 400 mg, and the like. The compressed pellet may include the cannabinoid resin in an amount greater than about 1 g. The cannabis composition pellet may include the cannabinoid resin present in any amount desired to be prescribed by the health practitioner. The compressed pellet may be one of a plurality of compressed pellets characterized by a standardized dose per pellet of the cannabinoid resin.

In some embodiments, the cannabinoid resin may be derived from a Cannabis crop. The Cannabis crop may be characterized by a seeded Cannabis crop. The Cannabis crop may be characterized by a short-season Cannabis crop. The Cannabis crop may be grown in the absence of one or more of added: fertilizers, pesticides, herbicides, fumigants, and the like.

In several embodiments, the cannabinoid resin may be derived from a Cannabis crop. The Cannabis crop may be characterized by a tetrahydrocannabinol content of less than about 5% (w/w) with respect to a dehydrated (dry) weight of the Cannabis crop. A dehydrated Cannabis crop may include a water content percentage (w/w) with respect to the dehydrated Cannabis crop of less than about one or more of: 2%, 1%, 0.9%, 0.8%, 0.7%, 0.6%, 0.5%, 0.4%, 0.3%, 0.2%, 0.1%, and 0.05%, or a range between any of the preceding values, for example, between about 0.2% and about 0.5%, or between about 0.4% and about 1%. The dehydrated Cannabis crop may be absent of water, e.g., less than about 0.05%, e.g., 0%. The cannabinoid resin may be derived from a Cannabis crop characterized by a tetrahydrocannabinol content with respect to a dehydrated weight of the Cannabis crop of less than about one or more of: 10%, 9%, 8%, 7%, 6%, 5%, 4%, 3%, 2%, and 1%, or a range between any of the preceding values, for example, between about 4% and about 8%, and the like.

Although various embodiments described herein are not limited to a cannabinoid resin derived from Cannabis crops including a specific tetrahydrocannabinol content, employing cannabinoid resins derived from Cannabis crops with a relatively low-tetrahydrocannabinol content may positively impact various aspects of regulation and production. For example, low-tetrahydrocannabinol-containing Cannabis crops may be easily and reliably grown in the absence of the extensive horticultural optimizations commonly employed to produce higher-tetrahydrocannabinol-containing Cannabis crops. Thus, low-tetrahydrocannabinol-containing Cannabis crops may be grown in open fields without burdensome optimization of growing conditions, chemical additives in the form of fertilizers, etc. Further, large-scale, open-field Cannabis crop production may alleviate the need for large numbers of growers. Limiting the need for many growers may decrease the efforts for regulation and compliance. Further, for example, low-tetrahydrocannabinol-containing Cannabis crops may be considerably less attractive to the illicit recreational market, and thus low-tetrahydrocannabinol-containing Cannabis crops may be grown with little or no monitoring against theft, contrary to high-tetrahydrocannabinol-containing Cannabis crops.

The nature of the amounts of the tetrahydrocannabinol extract and the cannabidiol extract in the cannabinoid resin may relate to the respective amounts present in the particular strain of Cannabis crop that the resin may be derived from. For example, a particular Cannabis crop may include a dominance in tetrahydrocannabinol, e.g., a tetrahydrocannabinol:cannabidiol ratio of about 3:1. Alternatively, a particular Cannabis crop may be bred to include a dominance in cannabidiol. Moreover, tetrahydrocannabinol and/or cannabidiol may be isolated and/or purified from the cannabinoid resin.

In some embodiments, the compressed pellet may include a pill, a capsule, a tablet, a soft-gel, a bead, and the like. The compressed pellet may be of any shape, such as oval, spherical, cylindrical, conical, cubic, rectangular, and the like. The shape of the compressed pellet may be designed to accommodate a device, such as a vaporizer, a pipe, a bong, a “oneie,” and the like.

In several embodiments, the compressed pellet may be configured for one or more of: ingestion, inhalation, and transdermal delivery. An ingestible compressed pellet may be swallowed or contacted with a mucous membrane for absorption over time. An ingestible compressed pellet may be one or more of: cooked, baked, and formulated in foodstuffs. For example, the compressed pellet may be included in foodstuffs of one or more of: cookies, brownies, cakes, bars, pastries, breads, hard and soft candies, syrups, sauces, and drinks. For example, the compressed pellet may be cooked in foodstuffs such as spaghetti.

In some embodiments, an inhalable compressed pellet may use a heating process to provide a cannabis vapor. The compressed pellet may be configured to be one or more of: at least partly, substantially, and completely vaporized at a temperature between about 100° C. and about 200° C. at a pressure of 1 atmosphere. In some embodiments, the compressed pellet may be used in a vaporizer or pipe. Alternatively, the compressed pellet may be rolled in cigarette paper. The compressed pellet may be heated on or in a device such that the vapor expands to at least partially fill a room. The substantial decrease in temperature to vaporize the compressed pellet may increase safety, as compared to smoking of the Cannabis plant material at 500° C.-700° C. At temperatures of 500° C.-700° C., many of the carcinogenic substances in the plant material may be burned and inhaled.

In several embodiments, the compressed pellet may be applied to a transdermal patch to be worn on the skin.

In some embodiments, the compressed pellet may include a casing. The casing may include any known pharmaceutical casing composition, for example, animal proteins such as gelatin, polysaccharides such as starch or cellulose, and the like. The compressed pellet may include or may be contacted with an anti-clumping medium. The anti-clumping medium may be effective to mitigate adhesion, clumping, or the like between two or more of the compressed pellets. The anti-clumping medium may be used in place of a casing or in addition to a casing, i.e., the anti-clumping medium may be a casing or a casing may be applied to the cannabinoid resin including the anti-clumping medium. The anti-clumping medium may include any anti-clumping agent known to the art, e.g., dry lubricants such as starch, edible wax, and the like, and preferably may include for example, hemp oil, fine (cannabis) leaf dust, and the like. The anti-clumping medium may include any material that coats the cannabinoid resin to reduce the stickiness of the cannabinoid resin.

In several embodiments, the compressed pellet may be packaged for dispensing. The packaging may house an individual compressed pellet in unit dose or a plurality of pellets in unit dosages. The packaging may include a bottle or container. The packaging may include a wrapper, e.g., bubble-card wrapping. The packaging may include a bandolier, e.g. overwrapping. The packaging may include a packet, a pouch, a cup, or a blister pack. The packaging may include any pharmaceutically-acceptable form of packaging for medical agents.

In various embodiments, a method 100 of preparing a cannabis composition in pellet form is provided. Method 100 may include 102 providing a Cannabis crop. Method 100 may include 104 extracting a cannabinoid resin from the Cannabis crop. The cannabinoid resin may include one or more of: a tetrahydrocannabinol extract and a cannabidiol extract. Method 100 may include 106 formulating the cannabinoid resin into one or more compressed pellets.

In some embodiments, method 100 may include dehydrating the Cannabis crop. The dehydrating may occur prior to extraction of the cannabinoid resin. The dehydrating may include one or more of: distillation, azeotropic distillation, reduced pressure, heating, desiccation, and lyophilization, i.e., freeze-drying. The heating may include oven-drying, sun-drying, or drum-drying. After dehydrating, the Cannabis crop may include a moisture content percentage (w/w) with respect to the Cannabis crop of less than about one or more of: 15%, 14%, 13%, 12%, 11%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, 0.05%, and 0%, or a range between any of the preceding values, for example, between about 0.2% and about 0.5%, between about 0.4% and about 1%, and the like. In some embodiments, the dehydrated Cannabis crop may be absent of water, e.g., less than 0.05%, e.g., 0%.

In several embodiments, the Cannabis crop may be characterized by a tetrahydrocannabinol content of less than about 5% (w/w) with respect to a dehydrated weight of the Cannabis crop. The cannabinoid resin may be derived from a Cannabis crop characterized by a tetrahydrocannabinol content (w/w) with respect to a dehydrated weight of the Cannabis crop of less than about one or more of: 10%, 9%, 8%, 7%, 6%, 5%, 4%, 3%, 2%, and 1%, or a range between any of the preceding values, for example, between about 4% and about 8%, and the like.

In some embodiments, method 100 may include growing the Cannabis crop. Growing the Cannabis crop may be in the absence of one or more of added: fertilizers, pesticides, herbicides, insecticides, and fumigants. The growing may be in an outdoor field. The growing may be in the absence of artificial lighting.

In several embodiments, the extracting may include chemical extraction. Chemical extraction may include submerging or washing the Cannabis crop in a solvent, i.e., solvent extraction. The solvent may include a hydrocarbon solvent, such as propane, hexane, and the like. The solvent may include supercritical or subcritical CO₂. The extraction may include one or more of: mechanical extraction, percolation, trituration, solvent extraction, supercritical solvent extraction, subcritical solvent extraction, vaporization and condensation, steam distillation and condensation, and sublimation and condensation. In other embodiments, the extracting may include mechanical extraction and will be further described below as mechanical separation.

In some embodiments, the formulating may include contacting the cannabinoid resin with an anti-clumping medium. The anti-clumping medium may include one or more of: fine leaf dust and hemp oil. The anti-clumping medium may include any material that coats the cannabinoid resin to reduce the stickiness of the cannabinoid resin. The formulating may include encasing the cannabinoid resin in a casing composition. The casing composition may include any pharmaceutical casing composition known. For example, the casing composition may include animal proteins such as gelatin, or polysaccharides such as starch or cellulose.

In several embodiments, the tetrahydrocannabinol extract may be present in the cannabinoid resin in an amount in % (w/w) with respect to the cannabinoid resin of about one or more of: 50, 55, 60, 65, 70, 75, 80, 85, 90, 95, 99, and 100, or a range between any of the preceding values, for example, between about 50 and about 100, between about 60 and about 75, and the like. The tetrahydrocannabinol extract may be present in the cannabinoid resin in less than about 50% (w/w) with respect to the cannabinoid resin. The tetrahydrocannabinol extract may be present in the cannabinoid resin in amount in % (w/w) with respect to the cannabinoid resin of about one or more of: 0, 5, 10, 15, 20, 25, 30, 35, 40, 45 and 50, or a range between any of the preceding values, for example, between about 0 and about 20, between about 35 and about 45, and the like. The cannabinoid resin may be absent of the tetrahydrocannabinol extract.

In some embodiments, the cannabinoid resin may include an amount of cannabidiol extract. The cannabidiol extract may be present in the cannabinoid resin in an amount in % (w/w) with respect to the cannabinoid resin of about one or more of: 0, 5, 10, 15, 20, 25, 30, 35, 40, 45 50, 55, 60, 65, 70, 75, 80, 85, 90, 95, 99, and 100, or a range between any of the preceding values, for example, between about 5 and about 50, between about 25 and about 75, and the like. The cannabinoid resin may be absent of cannabidiol extract.

In several embodiments, the cannabinoid resin may include a tetrahydrocannabinol extract or a cannabidiol extract. For example, the cannabinoid resin may include a tetrahydrocannabinol extract and not include a cannabidiol extract. For example, the cannabinoid resin may include a cannabidiol extract and not include a tetrahydrocannabinol extract. The amount of tetrahydrocannabinol extract may be any amount in % (w/w) with respect to cannabinoid resin as disclosed herein. The amount of cannabidiol extract may be any amount in % (w/w) with respect to cannabinoid resin as disclosed herein. For example, the cannabinoid resin may include an amount of the tetrahydrocannabinol extract between about 50% (w/w) and about 100% (w/w) with respect to the cannabinoid resin. Alternatively, for example, the cannabinoid resin may include an amount of the cannabidiol extract between about 1% (w/w) and about 100% (w/w) with respect to the cannabinoid resin.

In some embodiments, the cannabinoid resin may include a tetrahydrocannabinol extract and a cannabidiol extract. For example, the cannabinoid resin may include an amount of the tetrahydrocannabinol extract between about 50% (w/w) and about 99.9% (w/w) with respect to the cannabinoid resin, and include an amount of the cannabidiol extract between about 0.1% (w/w) and about 50% (w/w) with respect to the cannabinoid resin. The amount of the tetrahydrocannabinol extract and the amount of cannabidiol extract may be any amount in % (w/w) with respect to cannabinoid resin as disclosed herein, as long as the combined amounts of the tetrahydrocannabinol and the cannabidiol extracts do not exceed 100%. For example, the cannabinoid resin may include about 50% tetrahydrocannabinol extract and about 50% cannabidiol extract. For example, the cannabinoid resin may include about 60% tetrahydrocannabinol extract and about 20% cannabidiol extract.

In several embodiments, the compressed pellet may include the cannabinoid resin in an amount in milligrams (mg) of one or more of about: 25, 50, 100, 125, 150, 175, 200, 250, 300, 350, 400, 450, 500, 600, 700, 800, 900, 1000, 1100, 1200, 1300, 1400, 1500, 1600, 1700, 1800, 1900, and 2000, or a range between any of the preceding values, for example, between about 25 mg and about 2000 mg, between about 50 mg and about 500 mg, between about 500 mg and about 1000 mg, between about 200 mg and about 400 mg, and the like. The compressed pellet may include the cannabinoid resin in an amount greater than about 1 g. The cannabis composition pellet may include the cannabinoid resin present in any amount desired to be prescribed by the health practitioner. The compressed pellet may be one of a plurality of compressed pellets characterized by a standardized dose per pellet of the cannabinoid resin.

In various embodiments, a method 200 of separating a plant resin from a harvested plant material is provided. Method 200 may include 202 providing the harvested plant material. The harvested plant material may include the plant resin. Method 200 may include at 204 least partly dehydrating the harvested plant material. Method 200 may include 206 cooling the harvested plant material to a process temperature at or below a resin solidification temperature effective to provide the plant resin in a solidified state. At the process temperature, method 200 may include 208 forming the harvested plant material into a plant material aggregate. The plant material aggregate may include the solidified plant resin and plant matter. At the process temperature, method 200 may include 210 subjecting the plant material aggregate to mechanical separation conditions effective to separate at least a portion of the solidified plant resin from the plant matter.

In some embodiments, the harvested plant material may include plant components of one or more of: flowers, buds, seeds, stalks, roots, resin, and leaves. In some embodiments, method 200 may include removing the flowers from the harvested plant material prior to forming the harvested plant material into the plant material aggregate. In some embodiments, one or more plant components may include a relatively low-content of plant resin, e.g., less than about 10% (w/w) with respect to the harvested plant material.

In several embodiments, the harvested plant material may be derived from Cannabis crop. In many embodiments, the harvested plant material may include a plant resin. The plant resin may include a cannabinoid resin. The cannabinoid resin may include one or more of a tetrahydrocannabinol extract and a cannabidiol extract. The cannabinoid resin may include one or more of the tetrahydrocannabinol extract and the cannabidiol extract in any amounts described herein. For example, the cannabinoid resin may include one or more of: between about 50% and about 100% (w/w) of the tetrahydrocannabinol extract and between about 0.1% and about 100% (w/w) of the cannabidiol extract. For example, the cannabinoid resin may include between about 50% and about 99.9% of the tetrahydrocannabinol extract and between about 0.1% and about 50% of the cannabidiol extract. For example, the cannabidiol resin may include between about 50% (w/w) and about 100% (w/w) of the tetrahydrocannabinol extract with respect to the cannabinoid resin.

In some embodiments, the Cannabis crop may be characterized by any Cannabis crop described herein. For example, the Cannabis crop may be characterized by one or more of: a seeded Cannabis crop, a short-season Cannabis crop, and being grown in the absence of one or more of added: fertilizers, pesticides, herbicides, and fumigants.

In several embodiments, the Cannabis crop may be characterized by a tetrahydrocannabinol content of less than about 5% (w/w) with respect to a dehydrated weight of the Cannabis crop. The cannabinoid resin may be derived from a Cannabis crop characterized by a tetrahydrocannabinol content with respect to a dehydrated weight of the Cannabis crop of less than about one or more of: 10%, 9%, 8%, 7%, 6%, 5%, 4%, 3%, 2%, and 1%, or a range between any of the preceding values, for example, between about 4% and about 8%, and the like.

In some embodiments, method 200 may include at least partly dehydrating the harvested plant material. The dehydrating may result in a moisture percentage (w/w) with respect to the harvested plant material of less than about one or more of: 15%, 14%, 13%, 12%, 11%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, 0.05%, and 0.01%. The moisture percentage may refer to a water concentration. The dehydrating may result in a harvest plant material absent of water, e.g., 0%. In some embodiments, the dehydrating may include one or more of: distillation, azeotropic distillation, reduced pressure, heating, desiccation, and lyophilization. The heating may include oven-drying, sun-drying, or drum-drying.

In several embodiments, method 200 may include cooling the harvested plant material to a process temperature. The process temperature may be at or below a resin solidification temperature effective to provide the plant resin in a solidified state. The resin solidification temperature may include a value in ° C. of about one of: 10, 9, 8, 7, 6, 5, 4, 3, 2, 1, 0, −1, −2, −3, −4, −5, −6, −7, −8, −9, or −10. The process temperature may include a value in ° C. of about one of: 10, 9, 8, 7, 6, 5, 4, 3, 2, 1, 0, −1, −2, −3, −4, −5, −6, −7, −8, −9, −10, −11, −12, −13, −14, −15, −16, −17, −18, −19, −20, −21, −22, −23, −24, −25, −26, −27, −28, −29, or −30. The process temperature may include a temperature of less than about −3° C.

In some embodiments, method 200 may include forming the harvested plant material into a plant aggregate. The plant aggregate may include one or more of: flowers, buds, seeds, stalks, roots, resin, and leaves. The forming may include one or more of: grinding, chopping, macerating, blending, milling, pulverizing, pounding, and fracturing. The forming may occur at a process temperature. The process temperature may include any process temperature described herein. The forming may occur at a temperature above the process temperature, e.g., at about 20° C. to about 22° C. The plant material aggregate being formed may include one or more of: fibers, flakes, pieces, particulates, granules, and powder. The plant material aggregate may include a solidified plant resin and plant matter. The plant material aggregate may include a plant resin and plant matter. The plant material aggregate may be cooled to a process temperature after forming of the plant material aggregate to provide the solidified plant resin and plant matter.

In several embodiments, the solidified plant resin may be separated in a form that may include one or more of: flakes, pieces, granules, particulates, and powder. The solidified plant resin may be at least partly crystalline. The solidified plant resin may be at least partly glassy.

In some embodiments, the mechanical separation conditions may include separating the solidified plant resin from the plant matter according to one or more of: size, shape, density, and electrostatic charge. In some embodiments, the mechanical separation conditions may include separating the plant matter according to two or more of the: flowers, buds, seeds, stalks, roots, and leaves.

In several embodiments, the mechanical separation conditions may include separating the solidified plant resin from the plant matter using one or more of: filtration, gravitational or centrifugal settling, agitation, cyclonic separation, and electrostatic separation. The mechanical separation conditions may include separating the solidified plant resin from the plant matter by gravitational settling in the presence of mechanical vibration. The mechanical separation conditions may include separating the solidified plant resin from the plant matter using active settling in the presence of mechanical agitation. The mechanical agitation may include one or more of: stirring, shaking, vibrating, orbital oscillation, sonication, and gas fluidization. The method may include conducting the mechanical agitation using one or more of: a mechanical stirrer, a shaker table, a vibration unit, an orbital oscillation table, an ultrasound generator, and a gas injector.

In some embodiments, the conducting of the mechanical agitation may be for a period of time between about one or more of: 1 hour to 10 days, 6 hours to 10 days, 12 hours to 10 days, 1 to 10 days, 2 to 9 days, 3 to 8 days, 3 to 7 days, 3 to 6 days, and 4 to 5 days. The period of time for conducting the mechanical agitation may be any amount of time that may be effective to at least partly separate the solidified plant resin from the plant matter. The period of time for conducting the mechanical agitation may increase according to the scale of the separation.

In several embodiments, the mechanical separation conditions may include settling the solidified plant resin from the plant matter in a separation chamber. The separation chamber may include an elongated container. The separation chamber may be characterized by a length to a diameter. A ratio between the length to the diameter may include a range of one or more of: 3:1, 4:1, 5:1, 6:1, 7:1, 8:1, 9:1, and 10:1. The separation chamber may include an output at the bottom of the separation chamber with respect to a direction of the settling. The separation chamber may be characterized by a constant or decreasing cross-sectional area along a direction of the settling The constant or decreasing cross-sectional area may be in a plane perpendicular to the direction of the settling. The separation chamber may be characterized by a cross-sectional area in a plane perpendicular to a direction of the settling and a separation chamber interior surface area along the direction of the settling. A ratio between the cross-sectional area to the interior surface area may include a range of one or more of: 1:100 and 1:1, 1:75 and 1:1, 1:50 and 1:1, 1:40 and 1:2, 1:30 and 1:3. 1:20 and 1:4, and 1:15 and 1:5.

In some embodiments, the mechanical separation conditions may include cooling the separation chamber to a process temperature. The process temperature may be any process temperature described herein. The method may include maintaining an overall process temperature using one or more of: chilled water, ice, ice/water, ice/brine, a heat pump/refrigeration unit, and ambient temperature in the environment at or below the process temperature.

In several embodiments, the mechanical separation conditions may include batch separation. A batch separation may include loading the plant matter aggregate to the separation chamber, conducting the separation, stopping the separation, and removing at least a portion of one or more of the solidified plant resin and plant matter from the separation chamber. In other embodiments, the mechanical separation conditions may include continuous separation. A continuous separation may include loading the plant matter aggregate to the separation chamber, conducting the separation, removing at least a portion of one or more of the solidified plant resin and plant matter, and reloading the separation chamber with plant material aggregate without stopping the separation. In some embodiments, the mechanical separation conditions may include intermittent separation. Intermittent separation may include loading the plant aggregate material to the separation chamber, conducting the separation, reloading plant aggregate material to the separation chamber, further conducting separation, stopping the separation, and removing at least a portion of one or more of the solidified plant resin and plant matter. A single loading of plant aggregate material may undergo more than one round of separation cycles so as to increase yield of the separation.

In some embodiments, method 200 may include periodically or continuously removing one or more of: a separated portion of the solidified plant resin and a separating portion of the plant matter. The periodic or continuous removal may include one or more of: decanting, gravity flow, and scooping.

In several embodiments, method 200 may include periodically or continuously subjecting, i.e., loading, additional plant material aggregate to the mechanical separation conditions.

In some embodiments, the mechanical separation conditions may include an overall continuous operation. The overall continuous operation may include cooling to a process temperature, mechanically forming the plant material aggregate, mechanically loading the plant material aggregate to the separation chamber, separating the plant material aggregate into solidified plant resin and plant matter, and mechanically removing at least a portion of one or more of the solidified plant resin and plant matter.

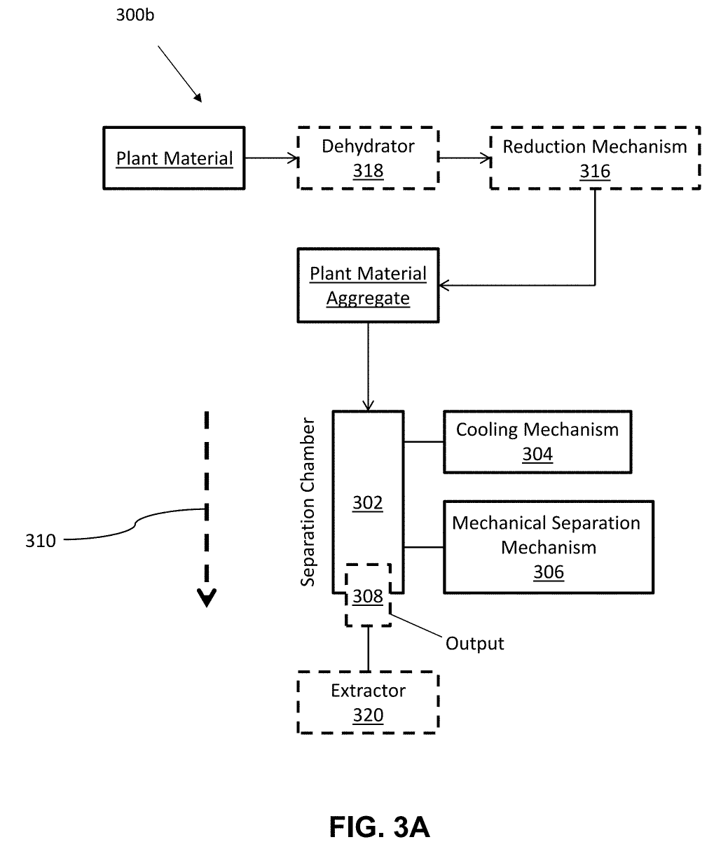

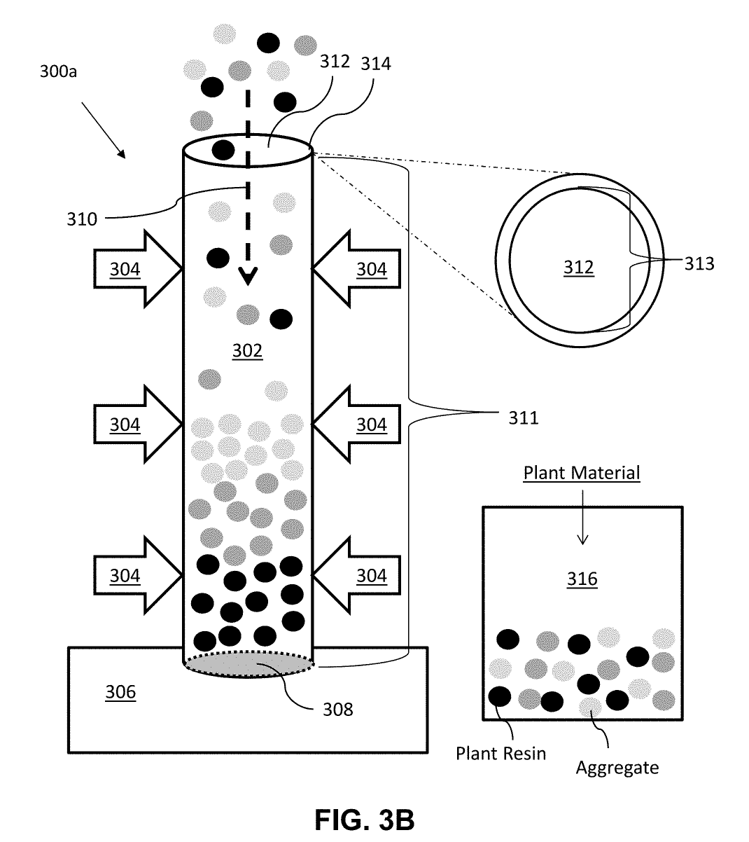

In various embodiments, an apparatus 300 for separating a plant resin from a harvested plant material is provided. FIG. 3A demonstrates various aspects of example apparatus 300 in view 300A. Schematic operation of example apparatus 300 is illustrated in view 300B in FIG. 3B.

Apparatus 300 may include a separation chamber 302. Apparatus 300 may include a cooling mechanism 304 operatively coupled to cool separation chamber 302 and contents thereof to a process temperature. Apparatus 300 may include a mechanical separation mechanism 306. Separation chamber 302 and mechanical separation mechanism 306 may be operatively coupled to separate a plant aggregate into a solidified plant resin and plant matter.

In some embodiments, separation chamber 302 may include an output 308 at the bottom of separation chamber 302 with respect to a direction of settling 310.

In several embodiments, separation chamber 302 may be in the form of an elongated container, such as a cylinder. Separation chamber 302 may be characterized by a length 311 to a diameter 313. A ratio between length 311 to diameter 313 may include a range of one or more of: 3:1, 4:1, 5:1, 6:1, 7:1, 8:1, 9:1, and 10:1. Separation chamber 302 may be characterized by a constant or decreasing diameter 313 along the direction of settling 310 and length 311, e.g., separation chamber 302 in the form of a cone or funnel (not shown).

In some embodiments, separation chamber 302 may be characterized by a constant or decreasing cross-sectional area 312 along the direction of settling 310, e.g., a decreasing cross-sectional area 312 as a cone or funnel (not shown). The constant or decreasing cross-sectional area 312 may be in a plane perpendicular to the direction of settling 310.

In several embodiments, separation chamber 302 may be characterized by cross-sectional area 312 in a plane perpendicular to the direction of settling 310 and a separation chamber interior surface area 314 along the direction of settling 310. A ratio between cross-sectional area 312 and interior surface area 314 may include a range of about one or more of: 1:100 and 1:1, 1:75 and 1:1, 1:50 and 1:1, 1:40 and 1:2, 1:30 and 1:3. 1:20 and 1:4, and 1:15 and 1:5.

In some embodiments, mechanical separation mechanism 306 may be configured effective to separate the solidified plant resin from the plant matter according to one or more of: size, shape, density, and electrostatic charge.

In several embodiments, mechanical separation mechanism 306 may include one or more of: a filter, a gravitational settling direction, a mechanical stirrer, a shaker table, a vibration unit, an orbital oscillation table, an ultrasound generator, a gas injector, a fluidized bed, a cyclonic separator, and an electrostatic separator.

In some embodiments, cooling mechanism 304 may include one or more of: a chilled fluid bath, a chilled fluid circulator, and a heat pump/refrigeration unit.

In several embodiments, cooling mechanism 304 may be configured to cool separation chamber 302 and contents thereof to the process temperature in ° C. of about one of: 10, 9, 8, 7, 6, 5, 4, 3, 2, 1, 0, −1, −2, −3, −4, −5, −6, −7, −8, −9, −10, −11, −12, −13, −14, −15, −16, −17, −18, −19, −20, −21, −22, −23, −24, −25, −26, −27, −28, −29, or −30. In some embodiments, cooling mechanism 304 may be configured to cool separation chamber 302 and contents thereof to the process temperature of less than about −3° C.

In some embodiments, apparatus 300 may include a reduction mechanism 316 (see FIG. 3A). Reduction mechanism 316 may be configured to form the harvested plant material into the plant material aggregate for input into separation chamber 302. The plant material aggregate may include the plant resin and plant matter. Reduction mechanism 316 may include one or more of: a grinder, a chopper, a macerator, a blender, a ball mill, a mortar and pestle, and a hammer and anvil. In many embodiments, reduction mechanism 316 may be configured to form the harvested plant material into the plant material aggregate including one or more of: fibers, flakes, pieces, particulates, granules, and powder.

In several embodiments, apparatus 300 may include a dehydrator 318 (see FIG. 3A). Dehydrator 318 may be configured to at least partly dehydrate the harvested plant material. Dehydrator 318 may include, for example, one or more of: a vacuum pump, a Schlenk line, a rotary evaporator, a distillation apparatus, a desiccator, an oven, and a heating plate. Dehydrator 318 may be configured to at least partly dehydrate the harvested plant material to a moisture percentage (w/w) of less than about one or more of: 15%, 14%, 13%, 12%, 11%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, 0.05%, and 0.01%. Dehydrator 318 may be configured to dehydrate the harvested plant material such that the harvest plant material may be absent of water, e.g., 0%.

In some embodiments, apparatus 300 may include an extractor 320 (see FIG. 3A). Extractor 320 may be effective to remove the solidified plant resin from the separation chamber upon separation from the plant matter in the plant aggregate. Alternatively, extractor 320 may be configured to remove the plant matter from the separation chamber upon separation from the solidified plant resin in the plant aggregate. Extractor 320 may be configured to remove the solidified plant resin and the plant matter from the separation chamber upon separation of the solidified plant resin from the plant matter.

In several embodiments, apparatus 300 may be configured for one or more of: batch separation, continuous separation, and intermittent separation.

In various embodiments, a method 400 of treatment is provided. Method 400 may include 402 providing a patient in need of treatment for a condition. Method 400 may include 404 providing a cannabis composition in the form of a compressed pellet. Method 400 may include 406 administering the compressed pellet to the patient effective to ameliorate one or more symptoms of the condition, whereby the condition may be treated.

In some embodiments, method 400 may include a treatment for a condition of one or more of: cancer, migraine, anorexia, cachexia, multiple sclerosis, spinal cord injury, neuropathy, rheumatoid arthritis, schizophrenia, epilepsy, stroke, head injury, inflammatory bowel disease, neuropathic pain, chronic, pain, muscle spasm, nausea, sleep disturbance, HIV/AIDS, diabetic neuropathy, and fibromyalgia. The condition for treatment may include any condition in which the patient experiences physical or emotional pain, uncomfortableness, or uneasiness. The condition for treatment may include, for example, anxiety or post-traumatic stress disorder.

In several embodiments, 406 administering may include ingesting the compressed pellet by the patient. Ingesting may include, for example, consuming the compressed pellet, e.g., swallowing.

In some embodiments, 406 administering may include causing the compressed pellet to at least partly vaporize to form a cannabis vapor. 406 Administering may further include contacting the patient with the cannabis vapor. The causing of the compressed pellet to at least partly vaporize to form a cannabis vapor may include contacting the compressed pellet with energy in the form of one or more of: heat, light, radiofrequency energy, and ultrasound. The causing of the compressed pellet to at least partly, substantially, or completely vaporize to form a cannabis vapor may include heating the compressed pellet. The heating of the compressed pellet may be at a temperature of between about 100° C. and about 200° C. at a pressure of 1 atmosphere.

In several embodiments, method 400 may include providing a plurality of the compressed pellets characterized by a standardized amount of the cannabinoid resin per each compressed pellet. The standardized amount of the cannabinoid resin per each compressed pellet may be effective to deliver the dose or a portion of the dose of the cannabinoid resin to the patient effective to ameliorate the one or more symptoms of the condition in the patient.

In some embodiments, method 400 may include determining a dose of a cannabinoid resin effective to ameliorate the one or more symptoms of the condition. Method 400 may include providing dosage instructions directing a user to provide the cannabis composition in the form of one or more compressed pellets in an amount effective to deliver the dose of the cannabinoid resin to the patient.

In several embodiments, method 400 may include vaporizing the cannabis composition in the form of one or more compressed pellets in an amount effective to deliver the dose of the cannabinoid resin to the patient. In some embodiments, the cannabis composition in the form of the compressed pellet may include a cannabinoid resin. The cannabinoid resin may include one or more of: between about 50% and about 100% (w/w) of a tetrahydrocannabinol extract and between about 0.1% and about 100% (w/w) of a cannabidiol extract. In some embodiments, the cannabinoid resin may include between about 50% and about 99.9% of the tetrahydrocannabinol extract and between about 0.1% and about 50% of the cannabidiol extract. In other embodiments, the cannabis composition in the form of the compressed pellet may include a cannabinoid resin including one or more of the tetrahydrocannabinol extract and the cannabidiol extract in any amount disclosed herein.

In several embodiments, the compressed pellet may include the cannabinoid resin in an amount in milligrams (mg) of one or more of about: 25, 50, 100, 125, 150, 175, 200, 250, 300, 350, 400, 450, 500, 600, 700, 800, 900, 1000, 1100, 1200, 1300, 1400, 1500, 1600, 1700, 1800, 1900, and 2000, or a range between any of the preceding values, for example, between about 25 mg and about 2000 mg, between about 50 mg and about 500 mg, between about 500 mg and about 1000 mg, between about 200 mg and about 400 mg, and the like. The compressed pellet may include the cannabinoid resin in an amount greater than about 1 g. The cannabis composition pellet may include the cannabinoid resin present in any amount desired to be prescribed by the health practitioner. The compressed pellet may be one of a plurality of compressed pellets characterized by a standardized dose per pellet of the cannabinoid resin. In some embodiments, the compressed pellet may consist essentially of the cannabinoid resin. In other embodiments, the compressed pellet may consist of the cannabinoid resin. In many embodiments, the compressed pellet may include any compressed pellet described herein, e.g., the compressed pellet may include one or more of an anti-clumping medium, and a casing.

In several embodiments, the cannabinoid resin may be derived from a Cannabis crop. The Cannabis crop may be characterized by any of the Cannabis crops described herein. For example, the Cannabis crop may include one or more of: a seeded Cannabis crop and a short-season Cannabis crop. The Cannabis crop may be grown in the absence of one or more of: fertilizer, pesticide, herbicide, insecticide, and fumigant. For example, the Cannabis crop may be characterized by a tetrahydrocannabinol content percentage (w/w) with respect to a dehydrated weight of the Cannabis crop of less than about one or more of: 10%, 9%, 8%, 7%, 6%, 5%, 4%, 3%, 2%, and 1%.

In some embodiments, method 400 may include providing one or more of the compressed pellets each in a unit dose packet, pouch, cup, container, or blister pack. Method 400 may include providing a plurality of the compressed pellets characterized by a standardized dose per pellet. Method 400 may include providing the compressed pellets in any form described herein.

In various embodiments, a kit 500 is provided. Kit 500 may include a cannabis composition 502. The cannabis composition may be in the form of a compressed pellet. Kit 500 may include instructions 504. Instructions 504 may direct a user to provide a patient in need of treatment for a condition. Instructions 504 may direct a user to administer the compressed pellet to the patient.

In some embodiments, the condition may include one or more of: cancer, migraine, anorexia, cachexia, multiple sclerosis, spinal cord injury, neuropathy, rheumatoid arthritis, schizophrenia, epilepsy, stroke, head injury, inflammatory bowel disease, neuropathic pain, chronic, pain, muscle spasm, nausea, sleep disturbance, HIV/AIDS, diabetic neuropathy, and fibromyalgia. The condition for treatment may include any condition in which the patient experiences physical or emotional pain, uncomfortableness, or uneasiness. The condition for treatment may include, for example, anxiety or post-traumatic stress disorder.

In several embodiments, instructions 504 may direct a user to administer one or more of the compressed pellets to the patient via ingestion. The ingestion may be effective to ameliorate one or more symptoms of the condition, whereby the condition may be treated.

In some embodiments, instructions 504 may direct a user to administer one or more of the compressed pellets to the patient via causing the compressed pellet to at least partly, substantially, or completely vaporize the compressed pellet to form a cannabis vapor. Instructions 504 may include directing a user to contact the patient with the cannabis vapor. The contacting to the cannabis vapor may be effective to ameliorate one or more symptoms of the condition, whereby the condition may be treated.

In several embodiments, instructions 504 may include instructions to cause the compressed pellet to at least partly vaporize to form the cannabis vapor in the form of one or more of: heat, light, radiofrequency energy, and ultrasound. Instructions 504 may include instructions for heating the compressed pellet to a temperature between about 100° C. and about 200° C.

In some embodiments, instructions 504 may include directing a user to determine a dose of a cannabinoid resin effective to ameliorate the one or more symptoms of the condition. Instructions 504 may include providing dosage instructions directing a user to provide the cannabis composition in the form of one or more compressed pellets in an amount effective to deliver the dose of the cannabinoid resin to the patient.

In several embodiments, instructions 504 may include directing a user to vaporize the cannabis composition in the form of one or more compressed pellets in an amount effective to deliver the dose of the cannabinoid resin to the patient.

In some embodiments, instructions 504 may include directing a user to provide a plurality of the compressed pellets characterized by a standardized amount of the cannabinoid resin per each compressed pellet. The standardized amount of the cannabinoid resin per each compressed pellet may be effective to deliver the dose or a portion of the dose of the cannabinoid resin to the patient effective to ameliorate the one or more symptoms of the condition in the patient.

In several embodiments, the cannabis composition in the form of the compressed pellet may include a cannabinoid resin including one or more of: between about 50% and about 100% (w/w) of a tetrahydrocannabinol extract and between about 0.1% and about 100% (w/w) of a cannabidiol extract. In some embodiments, the cannabinoid resin may include between about 50% and about 99.9% of the tetrahydrocannabinol extract and between about 0.1% and about 50% of the cannabidiol extract. In other embodiments, the cannabis composition in the form of the compressed pellet may include a cannabinoid resin including one or more of the tetrahydrocannabinol extract and the cannabidiol extract in any amount disclosed herein.

In several embodiments, the compressed pellet may consist essentially of the cannabinoid resin. In other embodiments, the compressed pellet may consist of the cannabinoid resin. In many embodiments, the compressed pellet may include any compressed pellet described herein, e.g., the compressed pellet may include one or more of an anti-clumping medium, and a casing. The anti-clumping medium may include one or more of: hemp oil and fine leaf dust.

In some embodiments, the compressed pellet may be configured to be at least partly, substantially, or completely vaporized between about 100° C. and about 200° C. at a pressure of 1 atmosphere.

In several embodiments, the compressed pellets may be characterized by a standardized dose per pellet.

In some embodiments, the cannabinoid resin may be derived from a Cannabis crop. The Cannabis crop may be characterized by any of the Cannabis crops described herein. For example, the Cannabis crop may include one or more of: a seeded Cannabis crop and a short-season Cannabis crop. The Cannabis crop may be grown in the absence of one or more of: fertilizer, pesticide, herbicide, insecticide, and fumigant. For example, the Cannabis crop may be characterized by a tetrahydrocannabinol content percentage (w/w) with respect to a dehydrated weight of the Cannabis crop of less than about one or more of: 10%, 9%, 8%, 7%, 6%, 5%, 4%, 3%, 2%, and 1%.

In several embodiments, kit 500 may include one or more of the compressed pellets each in a unit dose packet, pouch, cup, container, or blister pack. Kit 500 may include a plurality of the compressed pellets characterized by a standardized dose per pellet. Kit 500 may include the compressed pellets in any form described herein, e.g., packaging.

### EXAMPLES

### Prophetic Example 1

About 1 kg of a Cannabis crop may be harvested and optionally dehydrated by one or more of the dehydrating methods disclosed herein, e.g., lyophilization. The Cannabis crop may be submerged in a hydrocarbon solvent, e.g., hexane. The resulting mixture may optionally be heated to a temperature below the boiling point of the solvent, e.g., below 68° C. for hexane. A cannabinoid resin may be extracted from the Cannabis crop into the solvent. The mixture may be filtered and the solvent may be removed, e.g., evaporated under reduced pressure. The cannabinoid resin may be characterized for content and concentration via any known method, e.g., nuclear magnetic resonance (NMR) and/or high-performance liquid chromatography (HPLC). The cannabinoid resin may be portioned into standardized dosages based upon weight in view of the determined contents and concentrations. A cannabinoid resin obtained from one Cannabis crop may be combined with a cannabinoid resin from another Cannabis crop in order to reach the standardized dosage and desired contents and concentrations. The portioned cannabinoid resin may be contacted with an anti-clumping medium, e.g., dried leaf dust, and/or encased in a casing. The coated, portioned cannabinoid resin may be optionally cooled. The coated, portioned cannabinoid resin may be pressed into a compressed pellet of any shape.

### Prophetic Example 2

About 1 kg of a Cannabis crop may be harvested and dehydrated by one or more dehydrating methods disclosed herein, e.g., oven drying. The dehydrated Cannabis crop may be cooled to a temperature of about −3° C., or less, e.g., by submerging in a cooled bath or placed in a refrigeration unit. The cooled, dehydrated Cannabis crop may be subjected to a reduction mechanism, e.g., a grinder, to form a plant material aggregate. The plant material aggregate may include a solidified cannabinoid resin and plant matter. The plant material aggregate may be introduced into a cooled separation chamber. The separation chamber may be agitated by a separation mechanism, e.g., a shaker table or vibration unit. The plant material aggregate may separate in the separation chamber to form layers of the solidified cannabinoid resin and the plant matter. One or more of the solidified cannabinoid resin and the plant matter may be removed from the separation chamber via an extractor, e.g., gravity flow from an output. The solidified cannabinoid resin may be collected from the separation chamber and characterized for content and concentration via any known method, e.g., nuclear magnetic resonance (NMR) and/or high-performance liquid chromatography (HPLC). The solidified cannabinoid resin may be portioned into standardized dosages based upon weight in view of the determined contents and concentrations. A solidified cannabinoid resin obtained from one Cannabis crop may be combined with a solidified cannabinoid resin from another Cannabis crop in order to reach the standardized dosage and desired contents and concentrations. The portioned cannabinoid resin may be contacted with an anti-clumping medium, e.g., dried leaf dust, and/or encased in a casing. The coated, portioned cannabinoid resin may be pressed into a compressed pellet of any shape.

### Prophetic Example 3

A patient may be evaluated and diagnosed for a condition, e.g., cancer, and may have one or more associated symptoms, such as nausea, pain, cachexia, and the like. The patient may be prescribed a cannabis composition in the form of a compressed pellet to ameliorate one or more symptoms of the condition, e.g., pain. The prescription may include a recommended dosage of the cannabis composition. The recommended dosage of the cannabis composition may be based on one or more of: degree of severity of the symptoms, stage of the condition, and patient tolerance. The patient may be directed to ingest, e.g., swallow, one or more compressed pellets of the prescribe dosage at a prescribed interval of time.

### Prophetic Example 4

A patient may be evaluated and diagnosed for a condition, e.g., cancer. The patient having a condition may be prescribed a cannabis composition in the form of a compressed pellet to ameliorate one or more symptoms of the condition, e.g., pain. The prescription may include a recommended dosage of the cannabis composition. The recommended dosage of the cannabis composition may be based on one or more of: degree of severity of the symptoms, stage of the condition, and patient tolerance. The patient may be directed to ingest a food product formulated with one or more compressed pellets of the prescribed dosage at a prescribed interval of time. Alternatively, the patient may be directed to prepare a food product with one or more compressed pellets of the prescribed dosage at a prescribed interval of time.

### Prophetic Example 5

A patient may be evaluated and diagnosed for a condition, e.g., cancer. The patient having a condition may be prescribed a cannabis composition in the form of a compressed pellet to ameliorate one or more symptoms of the condition, e.g., pain. The prescription may include a recommended dosage of the cannabis composition. The recommended dosage of the cannabis composition may be based on one or more of: degree of severity of the symptoms, stage of the condition, and patient tolerance. The patient may be directed to vaporize the compressed pellet of the prescribed dosage, e.g., in a vaporizer device, and inhale the resulting cannabis vapor.

To the extent that the term “includes” or “including” is used in the specification or the claims, it is intended to be inclusive in a manner similar to the term “comprising” as that term is interpreted when employed as a transitional word in a claim. Furthermore, to the extent that the term “or” is employed (e.g., A or B) it is intended to mean “A or B or both.” When the applicants intend to indicate “only A or B but not both” then the term “only A or B but not both” will be employed. Thus, use of the term “or” herein is the inclusive, and not the exclusive use. See Bryan A. Garner, A Dictionary of Modern Legal Usage 624 (2d. Ed. 1995). Also, to the extent that the terms “in” or “into” are used in the specification or the claims, it is intended to additionally mean “on” or “onto.” To the extent that the term “selectively” is used in the specification or the claims, it is intended to refer to a condition of a component wherein a user of the apparatus may activate or deactivate the feature or function of the component as is necessary or desired in use of the apparatus. To the extent that the terms “operatively coupled” or “operatively connected” are used in the specification or the claims, it is intended to mean that the identified components are connected in a way to perform a designated function. To the extent that the term “substantially” is used in the specification or the claims, it is intended to mean that the identified components have the relation or qualities indicated with degree of error as would be acceptable in the subject industry.

As used in the specification and the claims, the singular forms “a,” “an,” and “the” include the plural unless the singular is expressly specified. For example, reference to “a compound” may include a mixture of two or more compounds, as well as a single compound.

As used herein, the term “about” in conjunction with a number is intended to include ±10% of the number. In other words, “about 10” may mean from 9 to 11.

As used herein, the terms “optional” and “optionally” mean that the subsequently described circumstance may or may not occur, so that the description includes instances where the circumstance occurs and instances where it does not.

As stated above, while the present application has been illustrated by the description of embodiments thereof, and while the embodiments have been described in considerable detail, it is not the intention of the applicants to restrict or in any way limit the scope of the appended claims to such detail. Additional advantages and modifications will readily appear to those skilled in the art, having the benefit of the present application. Therefore, the application, in its broader aspects, is not limited to the specific details, illustrative examples shown, or any apparatus referred to. Departures may be made from such details, examples, and apparatuses without departing from the spirit or scope of the general inventive concept.

The various aspects and embodiments disclosed herein are for purposes of illustration and are not intended to be limiting, with the true scope and spirit being indicated by the following claims.

1. A method 200 for separating a plant resin from a harvested plant material, comprising:
202 providing the harvested plant material, the harvested plant material comprising the plant resin;
204 at least partly dehydrating the harvested plant material;
206 cooling the harvested plant material to a process temperature at or below a resin solidification temperature effective to provide the plant resin in a solidified state;
at the process temperature:
208 forming the harvested plant material into a plant material aggregate, the plant material aggregate comprising the solidified plant resin and plant matter; and
210 subjecting the plant material aggregate to mechanical separation conditions effective to separate at least a portion of the solidified plant resin from the plant matter.

2. The method of claim 1, the harvested plant material being derived from a Cannabis crop, and the plant resin comprising a cannabinoid resin.

3. The method of claim 2, the cannabinoid resin comprising, one or more of:
between about 50% and about 100% (w/w) of a tetrahydrocannabinol extract; and
between about 0.1% and about 100% (w/w) of a cannabidiol extract.

4. The method of claim 3, the cannabinoid resin comprising, (w/w): between about 50% and about 99.9% of the tetrahydrocannabinol extract; and between about 0.1% and about 50% of the cannabidiol extract.

5. The method of claim 3, the cannabinoid resin comprising between about 50% (w/w) and about 100% (w/w) of the tetrahydrocannabinol extract with respect to the cannabinoid resin.

6. The method of claim 2, the Cannabis crop being characterized by one or more of: a seeded Cannabis crop, a short-season Cannabis crop, and being grown in the absence of one or more of added: fertilizer, pesticide, herbicide, insecticide, and fumigant.

7. The method of claim 2, the Cannabis crop being characterized by a tetrahydrocannabinol content percentage (w/w) with respect to a dehydrated weight of the Cannabis crop of less than about one or more of: 10%, 9%, 8%, 7%, 6%, 5%, 4%, 3%, 2%, and 1%.

8. The method of claim 1, comprising dehydrating the harvested plant material such that the harvested plant material is absent of water.

9. The method of claim 1, the dehydrating comprising one or more of: distillation, azeotropic distillation, reduced pressure, heating, desiccation, and lyophilization.

10. The method of claim 1, the resin solidification temperature being a value in ° C. of about one of: 10, 9, 8, 7, 6, 5, 4, 3, 2, 1, 0, −1, −2, −3, −4, −5, −6, −7, −8, −9, or −10.

11. The method of claim 1, the process temperature being a value in ° C. of about one of: 10, 9, 8, 7, 6, 5, 4, 3, 2, 1, 0, −1, −2, −3, −4, −5, −6, −7, −8, −9, −10, −11, −12, −13, −14, −15, −16, −17, −18, −19, −20, −21, −22, −23, −24, −25, −26, −27, −28, −29, or −30.

12. The method of claim 1, comprising forming the harvested plant material into the plant material aggregate by one or more of: grinding, chopping, macerating, blending, milling, pounding, pulverizing, and fracturing.

13. The method of claim 1, the plant material aggregate being formed comprising one or more of: fibers, flakes, pieces, granules, and powder.

14. The method of claim 1, the solidified plant resin being separated in a form comprising one or more of: flakes, pieces, granules, and powder.

15. The method of claim 1, the solidified plant resin being at least partly crystalline.

16. The method of claim 1, the solidified plant resin being at least partly glassy.

17. The method of claim 1, the mechanical separation conditions comprising separating the solidified plant resin from the plant matter according to one or more of: size, shape, density, and electrostatic charge.

18. The method of claim 1, the mechanical separation conditions comprising separating the solidified plant resin from the plant matter using one or more of: filtration, gravitational or centrifugal settling, agitation, cyclonic separation, and electrostatic separation.

19. The method of claim 1, the mechanical separation conditions comprising separating the solidified plant resin from the plant matter using active settling in the presence of mechanical agitation.

20. The method of claim 18, the mechanical agitation comprising one or more of: stirring, shaking, vibrating, orbital oscillation, sonication, and gas fluidization.

21. The method of claim 18, comprising conducting the mechanical agitation using one or more of: a mechanical stirrer, a shaker table, a vibration unit, an orbital oscillation table, an ultrasound generator, and a gas injector.

22. The method of claim 18, comprising conducting the mechanical agitation for a period of between about one or more of: 1 hour to 10 days, 6 hours to 10 days, 12 hours to 10 days, 1 to 10 days, 2 to 9 days, 3 to 8 days, 3 to 7 days, 3 to 6 days, and 4 to 5 days.

23. The method of claim 1, the mechanical separation conditions comprising separating the solidified plant resin from the plant matter by gravitational settling in the presence of mechanical vibration.

24. The method of claim 1, the mechanical separation conditions comprising settling the solidified plant resin from the plant matter in a separation chamber.

25. The method of claim 24, the separation chamber comprising an output at the bottom of the separation chamber with respect to a direction of the settling.

26. The method of claim 24, the separation chamber characterized by a constant or decreasing cross-sectional area along a direction of the settling, the constant or decreasing cross-sectional area being in a plane perpendicular to the direction of the settling.

27. The method of claim 24, the separation chamber characterized by a cross-sectional area in a plane perpendicular to a direction of the settling and a separation chamber interior surface area along the direction of the settling, a ratio between the cross-sectional area to the interior surface area being in a range of one or more of: 1:100 and 1:1, 1:75 and 1:1, 1:50 and 1:1, 1:40 and 1:2, 1:30 and 1:3. 1:20 and 1:4, and 1:15 and 1:5.

28. The method of claim 24, the separation chamber characterized by a length to a diameter, a ratio between the length to the diameter being in a range of one or more of: 3:1, 4:1, 5:1, 6:1, 7:1, 8:1, 9:1, and 10:1.

29. The method of claim 1, the mechanical separation conditions comprising batch separation, continuous separation, or intermittent separation.

30. The method of claim 1, comprising periodically or continuously removing one or more of: a separated portion of the solidified plant resin and a separated portion of the plant matter.

31. The method of claim 30, the periodic or continuous removal comprising one or more of decanting, gravity flow, and scooping.

32. The method of claim 1, comprising periodically or continuously subjecting additional plant material aggregate to the mechanical separation conditions.

33. The method of claim 1, the harvested plant material further comprising one or more of: flowers, buds, seeds, stalks, roots, and leaves.

34. The method of claim 33, further comprising separating from the harvested plant material one or more plant components having a comparatively low content of the plant resin.

35. The method of claim 33, further comprising separating the flowers from the harvested plant material prior to forming the harvested plant material into the plant material aggregate.

36. The method of claim 33, the mechanical separation conditions further separating the plant matter according to two or more of the: flowers, buds, seeds, stalks, roots, and leaves.

37. The method of claim 1, comprising cooling or maintaining the process temperature using one or more of: chilled water, ice, ice/water, ice/brine, a heat pump/refrigeration unit, and ambient temperature in an environment at or below the process temperature.

38. An apparatus 300 for separating a plant resin from a harvested plant material, comprising:
a separation chamber 302;
a cooling mechanism 304 operatively coupled to cool the separation chamber 302 and contents thereof to a process temperature; and
306 a mechanical separation mechanism;
the separation chamber 302 and the mechanical separation mechanism 306 being operatively coupled to separate a plant aggregate into a solidified plant resin and plant matter.

39. The apparatus of claim 38, the separation chamber 302 comprising an output 308 at a bottom of the separation chamber 302 with respect to a direction of settling 310.

40. The apparatus of claim 38, the separation chamber 302 being characterized by a length along a direction of settling 310 to a diameter perpendicular to the direction of settling 310, a ratio between the length to the diameter being in a range of between about one or more of: 3:1, 4:1, 5:1, 6:1, 7:1, 8:1, 9:1, and 10:1.

41. The apparatus of claim 38, the separation chamber 302 being characterized by a constant or decreasing cross-sectional area 312 along a direction of settling 310, the constant or decreasing cross-sectional area 312 being in a plane perpendicular to the direction of settling 310.

42. The apparatus of claim 38, the separation chamber 302 being characterized by a cross-sectional area 312 in a plane perpendicular to a direction of settling 310 and a separation chamber interior surface area 314 along the direction of settling 310, a ratio between the cross-sectional area 312 to the interior surface area 314 being in a range of one or more of: 1:100 and 1:1, 1:75 and 1:1, 1:50 and 1:1, 1:40 and 1:2, 1:30 and 1:3. 1:20 and 1:4, and 1:15 and 1:5.

43. The apparatus of claim 38, the mechanical separation mechanism 306 being configured effective to separate the solidified plant resin from the plant matter according to one or more of: size, shape, density, and electrostatic charge.

44. The apparatus of claim 38, the mechanical separation mechanism 306 comprising one or more of: a filter, a gravitational settling direction, a mechanical stirrer, a shaker table, a vibration unit, an orbital oscillation table, an ultrasound generator, a gas injector, a fluidized bed, a cyclonic separator, and an electrostatic separator.

45. The apparatus of claim 38, the cooling mechanism 304 comprising one or more of: a chilled fluid bath, a chilled fluid circulator, and a heat pump/refrigeration unit.

46. The apparatus of claim 38, the cooling mechanism 304 being configured to cool the separation chamber and contents thereof to the process temperature to a value in ° C. of about one of: 10, 9, 8, 7, 6, 5, 4, 3, 2, 1, 0, −1, −2, −3, −4, −5, −6, −7, −8, −9, −10, −11, −12, −13, −14, −15, −16, −17, −18, −19, −20, −21, −22, −23, −24, −25, −26, −27, −28, −29, or −30.

47. The apparatus of claim 38, the cooling mechanism 304 being configured to cool the separation chamber and contents thereof to the process temperature of less than about −3° C.

48. The apparatus of claim 38, further comprising a reduction mechanism 316 configured to form the harvested plant material into the plant material aggregate for input into the separation chamber 302, the plant material aggregate comprising the plant resin and the plant matter, the reduction mechanism comprising one or more of: a grinder, a chopper, a macerator, a blender, a ball mill, and hammer and anvil.

49. The apparatus of claim 48, the reduction mechanism 316 being configured to form the harvested plant material into the plant material aggregate comprising one or more of: fibers, flakes, pieces, granules, and powder.

50. The apparatus of claim 38, further comprising a dehydrator 318 configured to at least partly dehydrate the harvested plant material.

51. The apparatus of claim 38, being configured for batch separation, continuous separation, or intermittent separation.

52. The apparatus of claim 38, further comprising an extractor 320 configured to remove one or more of the solidified plant resin and the plant matter from the separation chamber upon separation of the solidified plant resin from the plant matter in the plant aggregate.
